SEQUENCE LISTING (9 sequences)

SEQ ID NO: 1 - protein, 1210 aa, Homo sapiens [sequence omitted]

SEQ ID NO: 2 - protein, 2839 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQATCHSLLNKATVKEKKENKKSVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGPFSKQTQIPDYAELIVKFLDALIDTYLPGIDEETSEESLLTPTSPYPPALQSQLSITANLNLSNSMTSLATSQHSPGIDKENVELSPTTGHCNSGRTRHGSASQVQKQRSAGSFKRNSIKKIV

SEQ ID NO: 3 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 4 - protein, 1786 aa, Homo sapiens
MGLLQLLAFSFLALCRARVRAQEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGNCFCYGHASECAPVDGFNEEVEGMVHGHCMCRHNTKGLNCELCMDFYHDLPWRPAEGRNSNACKKCNCNEHSISCHFDMAVYLATGNVSGGVCDDCQHNTMGRNCEQCKPFYYQHPERDIRDPNFCERCTCDPAGSQNEGICDSYTDFSTGLIAGQCRCKLNVEGEHCDVCKEGFYDLSSEDPFGCKSCACNPLGTIPGGNPCDSETGHCYCKRLVTGQHCDQCLPEHWGLSNDLDGCRPCDCDLGGALNNSCFAESGQCSCRPHMIGRQCNEVEPGYYFATLDHYLYEAEEANLGPGVSIVERQYIQDRIPSWTGAGFVRVPEGAYLEFFIDNIPYSMEYDILIRYEPQLPDHWEKAVITVQRPGRIPTSSRCGNTIPDDDNQVVSLSPGSRYVVLPRPVCFEKGTNYTVRLELPQYTSSDSDVESPYTLIDSLVLMPYCKSLDIFTVGGSGDGVVTNSAWETFQRYRCLENSRSVVKTPMTDVCRNIIFSISALLHQTGLACECDPQGSLSSVCDPNGGQCQCRPNVVGRTCNRCAPGTFGFGPSGCKPCECHLQGSVNAFCNPVTGQCHCFQGVYARQCDRCLPGHWGFPSCQPCQCNGHADDCDPVTGECLNCQDYTMGHNCERCLAGYYGDPIIGSGDHCRPCPCPDGPDSGRQFARSCYQDPVTLQLACVCDPGYIGSRCDDCASGYFGNPSEVGGSCQPCQCHNNIDTTDPEACDKETGRCLKCLYHTEGEHCQFCRFGYYGDALQQDCRKCVCNYLGTVQEHCNGSDCQCDKATGQCLCLPNVIGQNCDRCAPNTWQLASGTGCDPCNCNAAHSFGPSCNEFTGQCQCMPGFGGRTCSECQELFWGDPDVECRACDCDPRGIETPQCDQSTGQCVCVEGVEGPRCDKCTRGYSGVFPDCTPCHQCFALWDVIIAELTNRTHRFLEKAKALKISGVIGPYRETVDSVERKVSEIKDILAQSPAAEPLKNIGNLFEEAEKLIKDVTEMMAQVEVKLSDTTSQSNSTAKELDSLQTEAESLDNTVKELAEQLEFIKNSDIRGALDSITKYFQMSLEAEERVNASTTEPNSTVEQSALMRDRVEDVMMERESQFKEKQEEQARLLDELAGKLQSLDLSAAAEMTCGTPPGASCSETECGGPNCRTDEGERKCGGPGCGGLVTVAHNAWQKAMDLDQDVLSALAEVEQLSKMVSEAKLRADEAKQSAEDILLKTNATKEKMDKSNEELRNLIKQIRNFLTQDSADLDSIEAVANEVLKMEMPSTPQQLQNLTEDIRERVESLSQVEVILQHSAADIARAEMLLEEAKRASKSATDVKVTADMVKEALEEAEKAQVAAEKAIKQADEDIQGTQNLLTSIESETAASEETLFNASQRISELERNVEELKRKAAQNSGEAEYIEKVVYTVKQSAEDVKKTLDGELDEKYKKVENLIAKKTEESADARRKAEMLQNEAKTLLAQANSKLQLLKDLERKYEDNQRYLEDKAQELARLEGEVRSLLKDISQKVAVYSTCL

SEQ ID NO: 5 - protein, 494 aa, Homo sapiens
MFEIKKICCIGAGYVGGPTCSVIAHMCPEIRVTVVDVNESRINAWNSPTLPIYEPGLKEVVESCRGKNLFFSTNIDDAIKEADLVFISVNTPTKTYGMGKGRAADLKYIEACARRIVQNSNGYKIVTEKSTVPVRAAESIRRIFDANTKPNLNLQVLSNPEFLAEGTAIKDLKNPDRVLIGGDETPEGQRAVQALCAVYEHWVPREKILTTNTWSSELSKLAANAFLAQRISSINSISALCEATGADVEEVATAIGMDQRIGNKFLKASVGFGGSCFQKDVLNLVYLCEALNLPEVARYWQQVIDMNDYQRRRFASRIIDSLFNTVTDKKIAILGFAFKKDTGDTRESSSIYISKYLMDEGAHLHIYDPKVPREQIVVDLSHPGVSEDDQVSRLVTISKDPYEACDGAHAVVICTEWDMFKELDYERIHKKMLKPAFIFDGRRVLDGLHNELQTIGFQIETIGKKVSSKRIPYAPSGEIPKFSLQDPPNKKPKV

SEQ ID NO: 6 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 7 - protein, 105 aa, Homo sapiens
MAKISSPTETERCIESLIAVFQKYAGKDGYNYTLSKTEFLSFMNTELAAFTKNQKDPGVLDRMMKKLDTNSDGQLDFSEFLNLIGGLAMACHDSFLKAVPSQKRT

SEQ ID NO: 8 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 9 - protein, 393 aa, Homo sapiens [sequence omitted]

(19) United States
(12) Patent Application Publication
(10) Pub. No.: US 2023/0181499 A1
(43) Pub. Date: Jun. 15, 2023

(54) Hydroxyureamethyl-Acylfulvene for Treating Brain Cancer or CNS Cancer

(71) Applicant: Lantern Pharma Inc., Dallas, TX (US)

(72) Inventors: Aditya Kulkarni, Dallas, TX (US); Kishor Bhatia, Dallas, TX (US)

(21) Appl. No.: 17/995,904

(22) PCT Filed: Apr. 12, 2021
(86) PCT No.: PCT/US2021/026907

§ 371 Date: Oct. 10, 2022

Related U.S. Application Data
(60) Provisional application No. 63/008,359, filed on Apr. 10, 2020.

(51) Int. Cl. A61K 31/17; C12Q 1/6886; A61P 35/00; A61K 45/06

(52) U.S. Cl. CPC A61K 31/17; C12Q 1/6886; A61P 35/00; A61K 45/06; C12Q 2600/106; C12Q 2600/158; C12Q 2600/156

(57) ABSTRACT

Methods for treating brain tumor cancer by administration hydroxyureamethyl-acylfulvene. Some embodiments relate to treatment of glioblastoma by administration of hydroxyureamethyl-acylfulvene. Method also includes characterizing the subtype and genetics of the markers.

### TECHNICAL FIELD

This application relates to the field of chemistry and oncology. More particularly, this application relates to methods for treating brain cancer using hydroxyureamethyl-acylfulvene.

### BACKGROUND

Glioblastoma multiforme (GB, GBM or glioblastoma), with a median survival rate of around 15 months, ranks amongst the most aggressive of human cancers. The current standard of care for GB consists of de-bulking surgery followed by combined treatments with fractionated ionizing radiation (IR) and the DNA alkylating agent temozolomide (TMZ). The effectiveness of standard therapy with TMZ is limited because sensitivity of GBM to TMZ is dependent upon the methylation status of the DNA repair enzyme 06 Methyl Guanine Methyl Transferase (MGMT).

Glioblastoma is the most common and aggressive malignancy. Despite advances in therapy, improvement in overall survival has been limited. Patients with GB almost uniformly experience relapse and have a median survival time of only 15 to 20 months despite aggressive treatment with surgery, radiation, and chemotherapy. Surgical removal of the entire tumor is very difficult and impossible in most cases. Glioblastoma have finger-like tentacles that extend from the main tumor mass into surrounding normal brain tissue. Often surgical excision is limited by the balance between tumor removal and risks to cognitive function, or indeed immediate patient survival. GB can grow or metastasize to other areas in the brain.

Another reason GB is difficult to treat is that many drugs cannot efficiently cross the “blood-brain barrier” to enter the brain to act on the tumor. Glioblastomas comprise various populations of cells, some of which respond to treatment and others which do not. These other cells linger and spread through the brain, resulting in little long-term success. These and other factors make GB a pernicious and difficult tumor to characterize and treat.

Accordingly, there is always a need for an improved method and process for treating GB and other brain cancer.

### Definitions

Unless defined otherwise, all technical and scientific terms used herein have the same meaning as is commonly understood by one of skill in art to which the subject matter herein belongs. As used herein, the following definitions are supplied in order to facilitate the understanding of the present invention.

Amino acid sequence aligned with the amino acid sequence set out in SEQ ID NO: X (when referring to a variant polypeptide) means that the variant amino acid sequence and the amino acid sequence set out in SEQ ID NO: X are aligned by a suitable method which allows comparison of the sequences with each other and identifications of the positions in the amino acid sequence of the variant wherein either the same amino acid is present (identical position), or another amino acid is present (substitution), or one or more extra amino acids are present (insertion or extension) or no amino acid is present (deletion or truncation) if compared with the amino acid sequence set out in SEQ ID NO: X.

The term “antibody” as used herein includes intact molecules as well as molecules comprising or consisting of fragments thereof, such as, for example, Fab, F(ab′)2, Fv and scFv, as well as engineered variants including diabodies, triabodies, mini-bodies and single-domain antibodies which are capable of binding an epitopic determinant. Thus, antibodies may exist as intact immunoglobulins, or as modifications in a variety of forms.

The terms “patient,” “subject,” “individual,” and “host” refer to either a human or a non-human animal suffering from or suspected of suffering from a disease or disorder associated with aberrant biological or cell growth activity.

The term “biomarker” refers to any molecule, such as a gene, gene transcript (for example mRNA), peptide or protein or fragment thereof produced by a subject which is useful in differentiating subjects to predict the responsiveness of patients to treatments including disodium 2,2′-dithio-bis-ethane sulfonate or its analogs. A biomarker that is differentially present (i.e., increased or decreased) in a biological sample from a subject or a group of subjects has a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

The term “glioblastoma multiforme” or “GB” or “GBM” refers to the most common and aggressive type of primary brain tumor in humans. GB tumors are characterized by the presence of small areas of necrotizing tissue that is surrounded by anaplastic cells (pseudopalisading necrosis). This characteristic, as well as the presence of hyperplastic blood vessels, differentiates the tumor from Grade 3 astrocytomas, which do not have these features. In some cases, the term “glioblastoma multiforme” or “glioblastoma” “or malignant glioma” are used interchangeably herein and refer to a brain tumor that arises from astrocytes.

The terms “treat” and“treating” such a disease or disorder refers to ameliorating at least one symptom of the disease or disorder. These terms, when used in connection with a condition such as a cancer, refer to one or more of: impeding growth of the cancer, causing the cancer to shrink by weight or volume, extending the expected survival time of the patient, inhibiting tumor growth, reducing tumor mass, reducing size or number of metastatic lesions, inhibiting the development of new metastatic lesions, prolonging survival, prolonging progression-free survival, prolonging time to progression, and/or enhancing quality of life. The terms “treatment” or “treating” glioblastoma multiforme can include arresting the development or reversing the symptom or symptoms of glioblastoma and/or an improvement in clinical outcome of the patient suffering from glioblastoma or recurrent glioblastoma. Example of improvements in clinical outcome include longer survival time, reduction in tumor size, non-growth in tumor size, and/or lack of exacerbation in neurological symptoms. Non-limiting examples of neurological symptoms include double vision, vomiting, loss of appetite, changes in mood and personality, changes in ability to think and learn, seizures, speech difficulty, and cognitive impairment.

The term “preventing” when used in relation to a condition or disease such as cancer, refers to a reduction in the frequency of, or delay in the onset of, symptoms of the condition or disease. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The terms “expression level” and “level of expression,” as used herein, refer to the amount of a gene product in a cell, tissue, biological sample, organism, or patient, e.g., amounts of DNA, RNA (e.g. messenger RNA (mRNA)), or proteins corresponding to a given gene.

The term “pharmaceutically acceptable” means that, which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salt” refers to a salt which is acceptable for administration to a patient, such as a mammal (e.g., salts having acceptable mammalian safety for a given dosage regime). Such salts can be derived from pharmaceutically acceptable inorganic or organic bases and from pharmaceutically-acceptable inorganic or organic acids, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, N,N′-dibenzylethylenediamine, chloroprocaine, choline, diethanolamine, meglumine (N-methyl-glucamine) and the like.

When compounds of the present disclosure contain relatively basic functionalities, acid addition salts can be obtained by contacting the neutral form of such compounds with a sufficient amount of the desired acid, either neat or in a suitable inert solvent. Salts derived from pharmaceutically acceptable acids include acetic, trifluoroacetic, propionic, ascorbic, benzenesulfonic, benzoic, camphosulfonic, citric, ethanesulfonic, fumaric, glycolic, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, hydroiodic, carbonic, tartaric, p-toluenesulfonic, pyruvic, aspartic, benzoic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylaminosulfonic, algenic, hydroxybutyric, galactaric and galacturonic acid and the like.

The term “therapeutic effect” refers to a beneficial local or systemic effect in animals, particularly mammals, and more particularly humans, caused by administration of a compound or composition of the invention. The phrase “therapeutically-effective amount” means that amount of a compound or composition of the invention that is effective to treat a disease or condition caused by aberrant biological activity at a reasonable benefit/risk ratio. In some embodiments, the therapeutically effective amount of hydroxyureamethyl-acylfulvene or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

The term “healthy individual” shall be taken to mean an individual who is known not to suffer from cancer (e.g., brain cancer), such knowledge being derived from clinical data on the individual, including, but not limited to, a different diagnostic assay to that described herein.

A “reference level” means a level of the compound of the present invention or additional biomarker(s) that is indicative of a particular disease state, phenotype, or lack thereof, as well as combinations of disease states, phenotypes, or lack thereof.

A “reference sample” refers to a sample containing reference level of a biomarker. For example, a reference sample can be obtained from a subject that does not have a particular disease, disease state or phenotype, such as cancer or acute injury.

The therapeutically effective amount of such substance will vary depending upon the subject and disease condition being treated, the weight and age of the subject, the severity of the disease condition, the manner of administration and the like, which can readily be determined by one of skill in the art.

### SUMMARY

One aspect of this application includes a method of therapeutically treating brain cancer comprising administering an effective amount of hydroxyureamethyl-acylfulvene to a subject in need thereof. In one embodiment, the brain cancer is a glioblastoma multiforme.

Another aspect of this application includes a method for treating brain cancer using an effective amount of hydroxyureamethyl-acylfulvene together with an effective amount of one or more of an additional therapeutic agent selected from the group consisting of temozolomide, bevacizumab, everolimus, carmustine, lomustine, procarbazine, vincristine, irinotecan, cisplatin, carboplatin, methotrexate, etoposide, vinblastine, bleomycin, actinomycin, cyclophosphamide, and ifosfamide.

Another aspect of this application includes a method for treating brain cancer using an effective amount of hydroxyureamethyl-acylfulvene together with radiation therapy. The radiation therapy can be selected from whole-brain irradiation, fractionated radiotherapy, radio surgery, and a combination thereof.

Another aspect of this application includes a method in which the patient is human or animal.

### BRIEF DESCRIPTION OF THE DRAWINGS

FIG. 1 shows hydroxyureamethyl-acylfulvene exhibits nanomolar potency in GB cell lines;

FIG. 2 shows hydroxyureamethyl-acylfulvene exhibits nanomolar potency in additional GB cell lines;

FIG. 3 shows the analysis of the modulation of the blood-brain barrier by hydroxyureamethyl-acylfulvene;

FIG. 4 shows the impact on cell viability in the presence of hydroxyureamethyl-acylfulvene;

FIG. 5 shows the profile of hydroxyureamethyl-acylfulvene activity in vivo in the U87 subcutaneous (Sc) xenograft tumor model of GBM;

FIG. 6A shows results from mice implanted with GBM neurospheres (M1123) treated with a vehicle as a control;

FIG. 6B shows results from mice implanted with GBM neurospheres (M1123) treated with 4, every other day, intravenous doses of 4 mg/kg hydroxyureamethyl-acylfulvene.

FIG. 7 shows images of physical observations of mice implanted with GBM neurospheres (M1123) treated with vehicle control or hydroxyureamethyl-acylfulvene.

### SEQUENCES

SEQ ID NO:1—amino acid sequence of EGFR,

SEQ ID NO:2—amino acid sequence of NF1,

SEQ ID NO:3—amino acid sequence of IDH1,

SEQ ID NO:4—amino acid sequence of LAB1,

SEQ ID NO:5—amino acid sequence of IGDH,

SEQ ID NO:6—amino acid sequence of ANXA2,

SEQ ID NO:7—amino acid sequence of S100A11,

SEQ ID NO:8—amino acid sequence of CTSB,

SEQ ID NO:9—amino acid sequence of TP53, and

SEQ ID NO: 10—amino acid sequence of MGMT

### DETAILED DESCRIPTION

HydroxyUreaMethyl Acylfulvene or hydroxyureamethyl-acylfulvene (currently, termed as LP-184 by Lantern Pharma. Inc.) is a semisynthetic or synthetic antitumor agent derived from the mushroom toxin illudin S. The structure of each isomer is shown below.

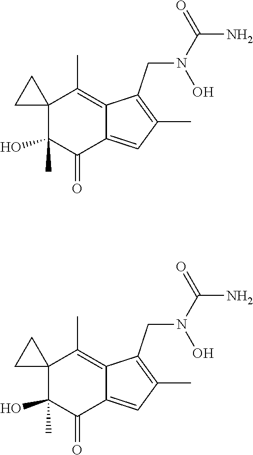 [SMILES: CC1=C(CN(O)C(N)=O)C2=C(C)C3(CC3)[C@@](C)(O)C(=O)C2=C1 ; CC1=C(CN(O)C(N)=O)C2=C(C)C3(CC3)[C@](C)(O)C(=O)C2=C1]

Specific embodiments relate to methods of treating brain cancer, the methods including the administration of an effective amount of hydroxyureamethyl-acylfulvene to a subject in need thereof. In some embodiments, the brain cancer may be from a metastatic brain tumor, glioblastoma multiforme, and a combination thereof. In specific examples, the brain tumor is a glioblastoma multiforme. In one example, hydroxyureamethyl-acylfulvene can be administered as a monotherapy.

One embodiment includes co-administering hydroxyureamethyl-acylfulvene and an additional therapeutic agent in separate compositions or the same composition. Thus, some embodiments include a first pharmaceutical composition comprising: (a) a safe and therapeutically effective amount of hydroxyureamethyl-acylfulvene or pharmaceutically acceptable salts thereof and (b) a pharmaceutically acceptable carrier, diluent, excipient or combination thereof, and a second pharmaceutical composition comprising: (1) a safe and therapeutically effective amount of an additional therapeutic agent and (2) a pharmaceutically acceptable carrier, diluent, excipient or combination thereof. Some embodiments include a pharmaceutical composition comprising: (a) a safe and therapeutically effective amount of an additional therapeutic agent; and (b) a pharmaceutically acceptable carrier, diluent, excipient or combination thereof. In some embodiments, the method described herein can further include subjecting the subject to a radiation therapy. In some embodiments, the radiation therapy can be a whole-brain irradiation, fractionated radiotherapy, and radiosurgery.

Another embodiment includes a method of inhibiting proliferation of a brain tumor cell, the method including contacting the brain tumor cell with hydroxyureamethyl-acylfulvene. In some embodiments, the contacting comprises administering an effective amount of hydroxyureamethyl-acylfulvene to a subject having the brain tumor cell. In some embodiments, the brain tumor is a glioblastoma multiforme. In some embodiments, the method can be used to treat primary Central Nervous System (CNS) tumors. CNS cancer is a type of cancer that forms in the central nervous system. It includes brain stem glioma, craniopharyngioma, medulloblastoma and meningioma. CNS tumors start in the normal cells of the brain and spinal cord called “neurons” and “glia.” Tumors that start from neurons include medulloblastoma and primitive neuroectodermal tumors (PNETs). Tumors that start from glia include glioma, astrocytoma, oligodendroglioma, and ependymoma. The tumor's specific name often reflects the CNS tumor's tissue of origin.

Another embodiment includes a method of therapeutically treating brain cancer or CNS tumors in a subject including obtaining a tumor sample from the subject; measuring the expression of MGMT+ or MGMT− in the tumor sample; and administering an effective amount of hydroxyureamethyl-acylfulvene or a pharmaceutically acceptable salt thereof to a subject in need thereof when MGMT+ measured to be greater than a reference, wherein the reference is the level of MGMT in a healthy person. In one example, the characterization further comprises a molecular subtype classification as a Classical, Mesenchymal, Proneural, or Neural glioblastoma.

Protein sequences of certain proteins identified herein include EGFR(SEQ ID NO: 1), NF1 (SEQ ID NO: 2), IDH1 (SEQ ID NO: 3), LAMB1 (SEQ ID NO: 4), UGDH (SEQ ID NO: 5), ANXA2 (SEQ ID NO: 6), S100A11 (SEQ ID NO: 7), CTSB (SEQ ID NO: 8), TP53 (SEQ ID NO: 9), and MGMT (SEQ ID NO: 10).

Some embodiments relate to a method of inducing apoptosis in a brain tumor cell, the method including contacting the brain tumor cell with hydroxyureamethyl-acylfulvene. In some embodiments, the contacting comprises administering an effective amount of hydroxyureamethyl-acylfulvene to a subject having the brain tumor cell. In some embodiments, the brain tumor is a glioblastoma multiforme.

Some embodiments include a treatment for GB that includes (1) conducting a physical exam of the patient; (2) using a magnetic resonance imaging (“MRI”) to confirm the presence of a tumor in the patient's brain: (3) conducting a brain biopsy to obtain genetic information about the cancer, which includes molecular subtype and genetic markers; and (4) prescribing effective treatment of hydroxyureamethyl-acylfulvene to a subject having GB.

The administration period can be a multi-week treatment cycle as long as the tumor remains under control and the regimen is clinically tolerated. In some embodiments, a single dosage of hydroxyureamethyl-acylfulvene or other therapeutic agent can be administered once a week, and preferably once on each of day 1 and day 8 of a three-week (21-day) treatment cycle. In some embodiments, a single dosage of hydroxyureamethyl-acylfulvene or other therapeutic agent can be administered once a week, twice a week, three times per week, four times per week, five times per week, six times per week, or daily during a one-week, two-week, three-week, four-week, or five-week treatment cycle. The administration can be on the same or different day of each week in the treatment cycle.

Another embodiment includes a method for treating or determining the sensitivity of brain cancer (e.g., Glioblastoma or CNS cancer) to a hydroxyureamethyl-acylfulvene treatment by assessing the level of MGMT expression. A level of MGMT greater than that in a healthy individual indicates that LP-184 can be effective.

A method of treating brain cancer or CNS cancer that includes detecting, in a human subject, the presence of certain genetic information; administering hydroxyureamethyl-acylfulvene or a pharmaceutically acceptable salt thereof, to the subject, if the human subject overexpress or under expresses certain markers. The marker can be substantially similar to EGFR (SEQ ID NO: 1), NF1 (SEQ ID NO: 2), IDH1 (SEQ ID NO: 3), LAMB1 (SEQ ID NO: 4), UGDH (SEQ ID NO: 5), ANXA2 (SEQ ID NO: 6), S100A11 (SEQ ID NO: 7), CTSB (SEQ ID NO: 8), TP53 (SEQ ID NO: 9), and MGMT (SEQ ID NO: 10).

Another embodiment includes a method for treating or determining the sensitivity of brain cancer (e.g., Glioblastoma) to a Hydroxyureamethyl-acylfulvene treatment by assessing the level of MGMT expression and/or at least one gene selected from the group consisting of: Laminin Subunit Beta 1 (LAMB1), UDP-Glucose 6-Dehydrogenase (UGDH), Annexin A2 (ANXA2), S100 Calcium Binding Protein A11 (S100A11), and Cathepsin B (CTSB). The nucleotide sequence and/or amino acid sequences are well known and published in public database (e.g., Genebank) and available papers.

Another embodiment includes a method of treating brain cancer in a subject, comprising: (a) obtaining or having obtained an expression level in a sample from a subject for a plurality of targets, wherein the plurality of targets comprises the group consisting of MGMT (SEQ ID NO: 10), LAMB1 (SEQ ID NO: 4), UGDH (SEQ ID NO: 5), ANXA2 (SEQ ID NO: 6), S100A11 (SEQ ID NO: 7), and/or CTSB (SEQ ID NO: 8); (b) determining that the subject is sensitive to a treatment with a Hydroxyureamethyl-acylfulvene; and (c) administering a cancer treatment including a Hydroxyureamethyl-acylfulvene.

Another embodiment includes a method of treating brain cancer in a subject, comprising: (a) obtaining or having obtained an expression level in a sample from a subject for a plurality of targets, wherein the plurality of targets comprises the group consisting of EGFR, NF1, and/or PDGFRA/IDH1: (b) determining that the subject is sensitive to a treatment with a Hydroxyureamethyl-acylfulvene; and (c) administering a cancer treatment including a Hydroxyureamethyl-acylfulvene.

Another embodiment includes the detection the genetic information including high-level EGFR amplification, deletions in NF1 gen, high expression of MET gene, alternations of PDFRA gene, point mutations in IDH1, TP53 mutation, expression of neuron markers by astrocytes, mutations in oncogenes, MGMT methylation, or a combination thereof.

Hydroxyureamethyl-acylfulvene for use in accordance with the present invention can be mainly administered by parenteral administration, specifically including subcutaneous administration, intramuscular administration, intravenous administration, transcutaneous administration, intrahecal administration, epidural administration, intra joint administration and local administration, or may also be administered in various dosage forms, for example by oral administration if possible.

The injections for parenteral administration include for example sterile, aqueous or non-aqueous solutions, suspensions and emulsions. The aqueous solutions and suspensions include for example distilled water for injections and physiological saline. The non-aqueous solutions and suspensions include for example propylene glycol, polyethylene glycol, vegetable oils such as olive oil, alcohols such as ethanol, and Polysorbate 80 (under trade name). Such composition may contain auxiliary agents such as preservatives, moistening agents, emulsifying agents, dispersing agents, stabilizers (for example, lactose) and dissolution auxiliary agents (for example, meglumine). These are sterilized by filtering through bacteria-retaining filters, blending sterilizing agents, or irradiation. Alternatively, these may be produced once into a sterile solid composition and then dissolved or suspended in sterile water or sterile solvents for injections, prior to use.

In some embodiments, the brain tumor can be selected from metastatic brain tumor, anaplastic astrocytoma, glioblastoma multiforme, oligodendroglioma, ependymomas, meningioma, mixed glioma, and a combination thereof. In some embodiments, the brain tumor is a glioblastoma multiforme. In some embodiments, the brain tumor is a metastatic brain tumor.

In some embodiments, the brain tumor can be selected from Anaplastic astrocytoma, Central neurocytoma, Choroid plexus carcinoma, Choroid plexus papilloma, Choroid plexus tumor, Dysembryoplastic neuroepithelial tumor, Ependymal tumor, Fibrillary astrocytoma, Giant-cell glioblastoma, Glioblastoma multiforme, Gliomatosis cerebri, Gliosarcoma, Hemangiopericytoma, Medulloblastoma, Medulloepithelioma, Meningeal carcinomatosis, Neuroblastoma, Neurocytoma, Oligoastrocytoma, Oligodendroglioma, Optic nerve sheath meningioma, Pediatric ependymoma, Pilocytic astrocytoma, Pinealoblastoma, Pineocytoma, Pleomorphic anaplastic neuroblastoma, Pleomorphic xanthoastrocytoma, Primary central nervous system lymphoma, Sphenoid wing meningioma, Subependymal giant cell astrocytoma, Subependymoma, central nervous system myeloma, and Trilateral retinoblastoma.

The liquid composition for oral administration includes for example pharmaceutically acceptable emulsions, liquids, suspensions, syrups and elixirs and contains inert diluents for general use, for example, distilled water and ethanol. The composition may contain auxiliary agents such as moistening agents and suspending agents, sweetening agents, flavoring agents, aromatic agents and preservatives, other than the inert diluents.

The active compound may also be administered intravenously or intraperitoneally by infusion or injection. Solutions of the active compound can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### EXAMPLES

### Example 1

Table 1 shows that LP-184/hydroxyureamethyl-acylfulvene shows enhanced sensitivity in tumors that express MGMT. In addition to MGMT expression, other GB-specific genes also show multi-omic parameter correlations with the possibility that LP-184/hydroxyureamethyl-acylfulvene will show sensitivity to GB.

Table 1 shows genes that can be used to show sensitivity of GB to hydroxyureamethyl-acylfulvene.

TABLE 1

| | Pearson Correlation Coefficient (R²) between LP-184 | | |
|---|---|---|---|
| | sensitivity and multi-omic parameter (p<0.05) | | |
| Upregulated | Transcript | Promoter | Protein |
| Gene | expression | Methylation | expression |
| MGMT | | −0.302 | |
| LPAR1 | 0.503 | −0.389 | |
| LAMB1 | 0.641 | −0.475 | 0.424 |
| SECTM1 | 0.34 | −0.323 | |
| UGDH | 0.477 | −0.373 | 0.41 |
| ZNF354A | 0.284 | | |
| VDR | | −0.312 | |

Table 2 shows genes downregulated in GB and associated with TMZ resistance that are negatively correlated with LP-184 sensitivity, and thus there can be a benefit of LP-184 in such tumors.

TABLE 2

| | Pearson Correlation Coefficient (R²) between | |
|---|---|---|
| Downregulated | LP-184 sensitivity and multi-omic parameter (p < 0.05) | |
| Gene | Transcript expression | Protein expression |
| HIST3H2A | −0.337 | |
| CYFIP2 | −0.374 | |
| MCM6 | −0.485 | |
| PDE4DIP | −0.308 | |
| PCMT1 | −0.315 | −0.423 |

Table 3 shows genes whose upregulation are known to promote GB. Tumors with elevated expression of such genes (compared to those in healthy people) can be more sensitive to hydroxyureamethyl-acylfulvene.

TABLE 3

| | Pearson Correlation Coefficient (R²) between LP-184 | | |
|---|---|---|---|
| Gene | sensitivity and multi-omic parameter (p < 0.05) | | |
| Promoting | Transcript | Promoter | Protein |
| GB | expression | Methylation | expression |
| EGFR | 0.638 | −0.554 | |
| ANXA2 | 0.553 | | 0.683 |
| S100A11 | 0.473 | −0.258 | 0.440 |
| APP | 0.746 | −0.531 | |
| DOCK1 | 0.659 | −0.624 | |
| G6PD | 0.378 | | |
| CARD10 | 0.544 | −0.561 | |
| CTSB | 0.318 | | 0.28 |

### Example 2

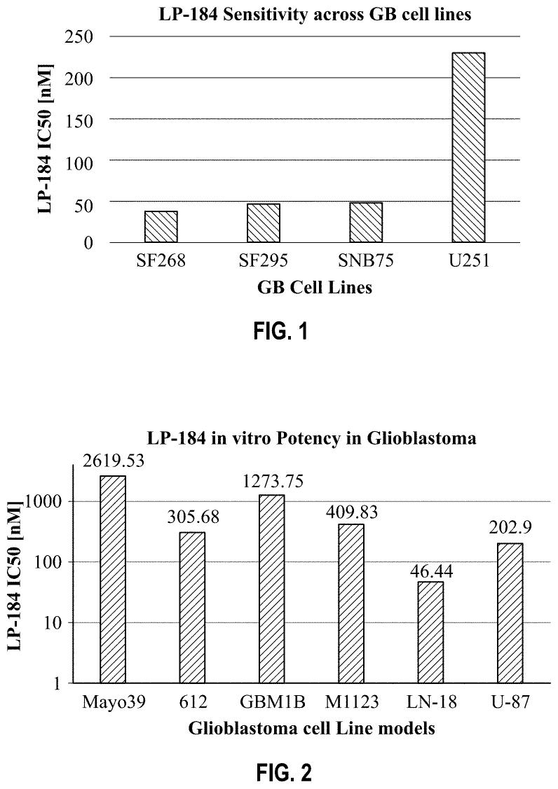

FIG. 1 shows that hydroxyureamethyl-acylfulvene exhibits nanomolar potency in GB cell lines. The cytotoxicity of LP-184 in terms of IC50 in the 4 cell lines is in the range of 45 to 230 nM.

The results from a tool for evaluating chemical ADMET (absorption, distribution, metabolism, excretion—toxicity) properties indicates that LP-184 crosses the blood brain barrier. NCI60 data generated drug sensitivity—gene expression correlation analysis of Hydroxyureamethyl-acylfulvene and Mitozolomide, a TMZ precursor molecule, showed that there are no overlapping genes that likely mediate resistance to either drug.

Hydroxyureamethyl-acylfulvene can serve as a potential monotherapy or as a combination therapy for GB tumors.

### Example 3

Hydroxyureamethyl-acylfulvene was tested and showed activity in an expanded spectrum of in vitro and in vivo brain cancer models that represent a variety of molecular and clinical subtypes of glioblastomas. Hydroxyureamethyl-acylfulvene was shown to cross the blood-brain barrier in an in vitro model system.

FIG. 2 shows that Hydroxyureamethyl-acylfulvene exhibits nanomolar potency in various GB cell lines beyond those shown in FIG. 1. The cytotoxicity of hydroxyureamethyl-acylfulvene in terms of IC50 in a set of 6 cell lines was in the range of 46 to 2620 nM.

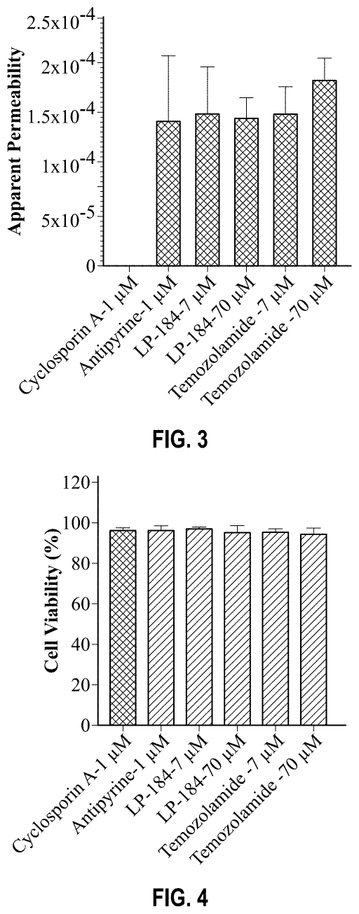

FIG. 3 shows the analysis of the modulation of the blood-brain barrier due to compound interaction. Hydroxyureamethyl-acylfulvene had insignificant impact on cell viability and blood-brain barrier integrity was not compromised. To evaluate the in vivo therapeutic activity of Hydroxyureamethyl-acylfulvene in mice bearing glioblastoma U87-SC cells (without any drug treatment in vitro) were inoculated subcutaneously.

By way of background, the physically, metabolically and immunologically privileged status of the brain presents diagnostic and therapeutic limitations along with treatment-related neurotoxicity issues. A critical property of any drug for GBM would be its ability to penetrate the blood-brain barrier (BBB). Independent in vitro studies assessing the penetration kinetics of Hydroxyureamethyl-acylfulvene showed Hydroxyureamethyl-acylfulvene passing across the blood-brain barrier. In the highly representative Neuromics in vitro 3D model that closely recapitulates the human blood-brain barrier, transport properties of the blood-brain barrier are reflected due to the formation of tight junctions, higher expression of specific carriers, or great cell viability. This 3D in vitro model of the blood-brain barrier created by co-culturing brain endothelial cells with pericytes and astrocytes layered in an insert, improves endothelial cell polarization and enhances the formation of tight junctions, provides better endothelial cell-to-cell contact that is important for barrier development, and prevents the dilution of secreted neurotrophic factors. These conditions collectively lead to the development of an in vitro model that can truly mimic the blood-brain barrier. This assay leverages the novel 3D blood-brain barrier model that allows studying both compound transport across the barrier as well as the effect of compounds on the structure and function of the blood-brain barrier. Along with LP-184 and TMZ, the current standard of care chemotherapeutic for GBM, we also compared the behavior of known positive and negative control agents Antipyrine and Cyclosporin A respectively as benchmarks. LP-184 is as effective as TMZ in penetrating the blood-brain barrier. Apparent blood brain barrier permeability measured for TMZ was 1.72*10-4 cm/s at 30 minutes and for LP-184 was 1.53*10-4 cm/s at 30 minutes, as illustrated in the following graph.

FIG. 4 shows the impact on cell viability after treatment with hydroxyureamethyl-acylfulvene.

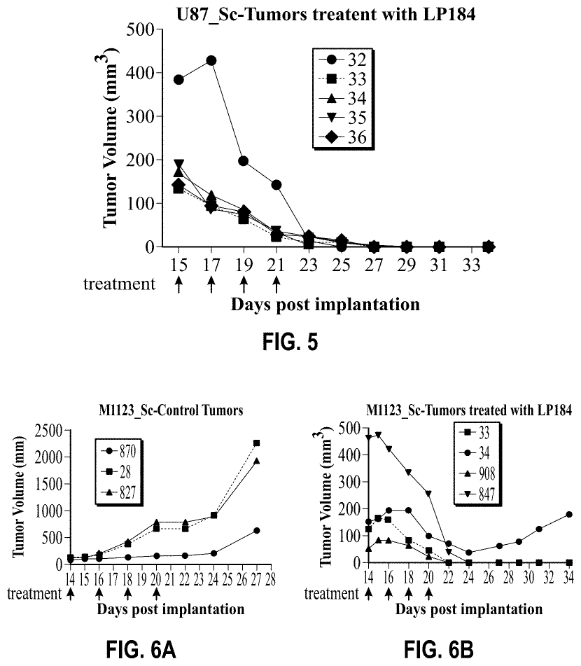

FIG. 5 shows the profile of hydroxyureamethyl-acylfulvene activity in vivo in the U87-Sc xenograft tumor model of GB. The mice were treated with 4, every other day, intravenous doses of Hydroxyureamethyl-acylfulvene at 4 mg/kg in the U87-SC model resulted in complete tumor regression with no measurable tumors 12 days post the last dosing in all 5 mice treated.

FIG. 6A and FIG. 6B shows that mice (M1123) treated with 4, every other day, intravenous doses of hydroxyureamethyl-acylfulvene at 4 mg/kg resulted in complete tumor regression. FIG. 6A shows results from mice implanted with GBM neurospheres (M1123) treated with a vehicle (control). FIG. 6B shows results from mice implanted with GBM neurospheres (M1123) treated with 4 every other day intravenous doses of 4 mg/kg hydroxyureamethyl-acylfulvene. Three of four mice showed no measurable tumor after 12 days of the last dose.

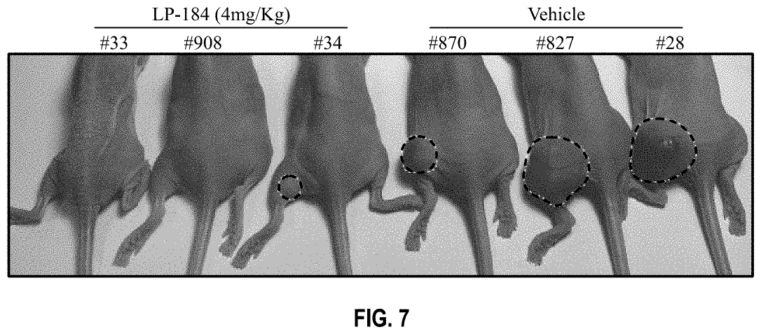

FIG. 7 shows images of physical observations of mice implanted with GBM neurospheres (M1123) treated with vehicle control or hydroxyureamethyl-acylfulvene. The tumor volume physically displayed clear tumor growth inhibition in Hydroxyureamethyl-acylfulvene treated mice relative to vehicle-treated control mice. This example shows in vivo efficacy of Hydroxyureamethyl-acylfulvene in subcutaneous tumor models of GB.

```
Protein sequences of EGFR(SEQ ID NO: 1),
NF1 (SEQ ID NO: 2), IDH1 (SEQ ID NO: 3),
LAMB1 (SEQ ID NO: 4), UGDH (SEQ ID NO: 5),
  ANXA2 (SEQ ID NO: 6), S100A11
(SEQ ID NO: 7), CTSB (SEQ ID NO: 8), TP53
(SEQ ID NO: 9), and MGMT (SEQ ID NO: 10)
  from Uniprot database:
SEQUENCE ID NO: 1
(SEQ ID NO: 1)
EGFR/Homo sapiens/Human
MRPSGTAGAALLALLAALCPASRALEEKKVCQGTSNKLTQLGTFEDHFLSLQRMFN
NCEVVLGNLEITYVQRNYDLSFLKTIQEVAGYVLIALNTVERIPLENLQIIRGNMYYE
NSYALAVLSNYDANKTGLKELPMRNLQEILHGAVRFSNNPALCNVESIQWRDIVSSD
FLSNMSMDFQNHLGSCQKCDPSCPNGSCWGAGEENCQKLTKIICAQQCSGRCRGKS
PSDCCHNQCAAGCTGPRESDCLVCRKFRDEATCKDTCPPLMLYNPTTYQMDVNPEG
KYSFGATCVKKCPRNYVVTDHGSCVRACGADSYEMEEDGVRKCKKCEGPCRKVCN
GIGIGEFKDSLSINATNIKHFKNCTSISGDLHILPVAFRGDSFTHTPPLDPQELDILKTV
KEITGFLLIQAWPENRTDLHAFENLEIIRGRTKQHGQFSLAVVSLNITSLGLRSLKEISD
GDVIISGNKNLCYANTINWKKLFGTSGQKTKIISNRGENSCKATGQVCHALCSPEGC
WGPEPRDCVSCRNVSRGRECVDKCNLLEGEPREFVENSECIQCHPECLPQAMNITCT
GRGPDNCIQCAHYIDGPHCVKTCPAGVMGENNTLVWKYADAGHVCHLCHPNCTYG
CTGPGLEGCPTNGPKIPSIATGMVGALLLLLVVALGIGLFMRRRHIVRKRTLRRLLQE
RELVEPLTPSGEAPNQALLRILKETEFKKIKVLGSGAFGTVYKGLWIPEGEKVKIPVAI
KELREATSPKANKEILDEAYVMASVDNPHVCRLLGICLTSTVQLITQLMPFGCLLDY
VREHKDNIGSQYLLNWCVQIAKGMNYLEDRRLVHRDLAARNVLVKTPQHVKITDF
GLAKLLGAEEKEYHAEGGKVPIKWMALESILHRIYTHQSDVWSYGVTVWELMTFGS
KPYDGIPASEISSILEKGERLPQPPICTIDVYMIMVKCWMIDADSRPKFRELIIEFSKMA
RDPQRYLVIQGDERMHLPSPTDSNFYRALMDEEDMDDVVDADEYLIPQQGFFSSPST
SRTPLLSSLSATSNNSTVACIDRNGLQSCPIKEDSFLQRYSSDPTGALTEDSIDDTFLPV
PEYINQSVPKRPAGSVQNPVYHNQPLNPAPSRDPHYQDPHSTAVGNPEYLNTVQPTC
VNSTFDSPAHWAQKGSHQISLDNPDYQQDFFPKEAKPNGIFKGSTAENAEYLRVAPQ
SSEFIGA
SEQUENCE ID NO: 2
(SEQ ID NO: 2)
NF1/Homo sapiens/Human
MAAHRPVEWVQAVVSRFDEQLPIKTGQQNTHTKVSTEHNKECLINISKYKFSLVISG
LTT
ILKNVNNMRIFGEAAEKNLYLSQLIILDTLEKCLAGQPKDTMRLDETMLVKQLLPEIC
HF
LHTCREGNQHAAELRNSASGVLFSLSCNNFNAVFSRISTRLQELTVCSEDNVDVHDIE
LL
QYINVDCAKLKRLLKETAFKFKALKKVAQLAVINSLEKAFWNWVENYPDEFTKLYQ
IPQTDMAECAEKLFDLVDGFAESTKRKAAVWPLQIILLILCPEIIQDISKDVVDENNM
NKKLFLDSLRKALAGHGGSRQLTESAAIACVKLCKASTYINWEDNSVIFLLVQSMVV
DLKNLLFNPSKPFSRGSQPADVDLMIDCLVSCFRISPHNNQHFKICLAQNSPSTFHYV
LVNSLHRIITNSALDWWPKIDAVYCHSVELRNMFGETLHKAVQGCGAHPAIRMAPS
LTFKEKVTSLKFKEKPTDLETRSYKYLLLSMVKLIHADPKLLLCNPRKQGPETQGST
AELITGLVQLVPQSHMPEIAQEAMEALLVLHQLDSIDLWNPDAPVETFWEISSQMLF
YICKKLTSHQMLSSTEILKWLREILICRNKFLLKNKQADRSSCHFLLFYGVGCDIPSSG
NTSQMSMDHEELLRTPGASLRKGKGNSSMDSAAGCSGTPPICRQAQTKLEVALYMF
LWNPDTEAVLVAMSCFRHLCEEADIRCGVDEVSVHNLLPNYNTFMEFASVSNMMST
GRAALQKRVMALLRRIEHPTAGNTEAWEDTHAKWEQATKLILNYPKAKMEDGQAA
ESLHKTTVKRRMSHVSGGGSIDLSDTDSLQEWINMTGFLCALGGVCLQQRSNSGLAT
YSPPMGPVSERKGSMISVMSSEGNADTPVSKFMDRLLSLMVCNHEKVGLQIRTNVK
DLVGLELSPALYPMLFNKLKNTISKFFDSQGQVLLTDTNTQFVEQTIAIMKNLLDNHT
EGSSEHLGQASIETMMLNLVRYVRVLGNMVHAIQIKTKLCQLVEVMMARRDDLSFC
QEMKFRNKMVEYLTDWVMGTSNQAADDDVKCLTRDLDQASMEAVVSLLAGLPLQ
PEEGDGVELMEAKSQLFLKYFTLFMNLLNDCSEVEDESAQTGGRKRGMSRRLASLR
HCTVLAMSNLLNANVDSGLMHSIGLGYHKDLQTRATFMEVLTKILQQGTEFDTLAE
TVLADRFERLVELVTMMGDQGELPIAMALANVVPCSQWDELARVLVTLFDSRHLL
YQLLWNMFSKEVELADSMQTLFRGNSLASKIMTFCFKVYGATYLQKLLDPLLRIVIT
SSDWQHVSFEVDPTRLEPSESLEENQRNLLQMTEKFFHAIISSSSEFPPQLRSVCHCLY
QATCHSLLNKATVKEKKENKKSVVSQRFPQNSIGAVGSAMFLRFINPAIVSPYEAGIL
DKKPPPRIERGLKLMSKILQSIANHVLFTKEEHMRPFNDFVKSNFDAARRFFLDIASD
CPTSDAVNHSLSFISDGNVLALHRLLWNNQEKIGQYLSSNRDHKAVGRRPFDKMAT
LLAYLGPPEHKPVADTHWSSLNLTSSKFEEFMTRHQVHEKEEFKALKTLSIFYQAGT
SKAGNPIFYYVARRFKTGQINGDLLIYHVLLTLKPYYAKPYEIVVDLTHTGPSNRFKT
DFLSKWFVVFPGFAYDNVSAVYIYNCNSWVREYTKYHERLLTGLKGSKRLVFIDCP
GKLAEHIEHEQQKLPAATLALEEDLKVFHNALKLAHKDTKVSIKVGSTAVQVTSAER
TKVLGQSVFLNDIYYASEIEEICLVDENQFTLTIANQGTPLTFMHQECEAIVQSIIHIRT
RWELSQPDSIPQHTKIRPKDVPGTLLNIALLNLGSSDPSLRSAAYNLLCALTCTFNLKI
EGQLLETSGLCIPANNTLFIVSISKTLAANEPHLTLEFLEECISGFSKSSIELKHLCLEYM
TPWLSNLVRFCKHNDDAKRQRVTAILDKLITMTINEKQMYPSIQAKIWGSLGQITDL
LDVVLDSFIKTSATGGLGSIKAEVMADTAVALASGNVKLVSSKVIGRMCKIIDKTCLS
PTPTLEQHLMWDDIAILARYMLMLSFNNSLDVAAHLPYLFHVVTFLVATGPLSLRAS
THGLVINIIHSLCTCSQLHFSEETKQVLRLSLTEFSLPKFYLLFGISKVKSAAVIAFRSS
YRDRSFSPGSYERETFALTSLETVTEALLEIMEACMRDIPTCKWLDQWTELAQRFAF
QYNPSLQPRALVVFGCISKRVSHGQIKQIIRILSKALESCLKGPDTYNSQVLIEATVIAL
TKLQPLLNKDSPLHKALFWVAVAVLQLDEVNLYSAGTALLEQNLHTLDSLRIFNDK
SPEEVFMAIRNPLEWHCKQMDHFVGLNFNSNFNFALVGHLLKGYRHPSPAIVARTV
RILHTLLTLVNKHRNCDKFEVNTQSVAYLAALLTVSEEVRSRCSLKHRKSLLLTDIS
MENVPMDTYPIHHGDPSYRTLKETQPWSSPKGSEGYLAATYPTVGQTSPRARKSMS
LDMGQPSQANTKKLLGTRKSFDHLISDTKAPKRQEMESGITTPPKMRRVAETDYEM
ETQRISSSQQHPHLRKVSVSESNVLLDEEVLTDPKIQALLLTVLATLVKYTTDEFDQRI
LYEYLAEASVVFPKVFPVVHNLLDSKINTLLSLCQDPNLLNPIHGIVQSVVYHEESPP
QYQTSYLQSFGFNGLWRFAGPFSKQTQIPDYAELIVKFLDALIDTYLPGIDEETSEESL
LTPTSPYPPALQSQLSITANLNLSNSMTSLATSQHSPGIDKENVELSPTTGHCNSGRTR
HGSASQVQKQRSAGSFKRNSIKKIV
SEQUENCE ID NO: 3
(SEQ ID NO: 3)
IDH1/Homo sapiens/Human
MSKKISGGSVVEMQGDEMTRIIWELIKEKLIFPYVELDLHSYDLGIENRDATNDQVT
KDAAEAIKKHNVGVKCATITPDEKRVEEFKLKQMWKSPNGTIRNILGGTVFREAIIC
KNIPRLVSGWVKPIIIGRHAYGDQYRATDFVVPGPGKVEITYTPSDGTQKVTYLVHN
FEEGGGVAMGMYNQDKSIEDFAHSSFQMALSKGWPLYLSTKNTILKKYDGRFKDIF
QEIYDKQYKSQFEAQKIWYEHRLIDDMVAQAMKSEGGFIWACKNYDGDVQSDSVA
QGYGSLGMMTSVLVCPDGKTVEAEAAHGTVTRHYRMYQKGQETSTNPIASIFAWT
RGLAHRAKLDNNKELAFFANALEEVSIETIEAGFMTKDLAACIKGLPNVQRSDYLNT
FEFMDKLGENLKIKLAQAKL
SEQUENCE ID NO: 4
(SEQ ID NO: 4)
LAMB1/Homo sapiens/Human
MGLLQLLAFSFLALCRARVRAQEPEFSYGCAEGSCYPATGDLLIGRAQKLSVTSTCG
LHKPEPYCIVSHLQEDKKCFICNSQDPYHETLNPDSHLIENVVTTFAPNRLKIWWQSE
NGVENVTIQLDLEAEFHFTHLIMTFKTFRPAAMLIERSSDFGKTWGVYRYFAYDCEA
SFPGISTGPMKKVDDIICDSRYSDIEPSTEGEVIFRALDPAFKIEDPYSPRIQNLLKITNL
RIKFVKL
HTLGDNLLDSRMEIREKYYYAVYDMVVRGNCFCYGHASECAPVDGFNEEVEGMVH
GHCMCRHNTKGLNCELCMDFYHDLPWRPAEGRNSNACKKCNCNEHSISCHFDMAV
YLATGNVSGGVCDDCQHNTMGRNCEQCKPFYYQHPERDIRDPNFCERCTCDPAGSQ
NEGICDSYTDFSTGLIAGQCRCKLNVEGEHCDVCKEGFYDLSSEDPFGCKSCACNPL
GTIPGGNPCDSETGHCYCKRLVTGQHCDQCLPEHWGLSNDLDGCRPCDCDLGGALN
NSCFAESGQCSCRPHMIGRQCNEVEPGYYFATLDHYLYEAEEANLGPGVSIVERQYI
QDRIPSWTGAGFVRVPEGAYLEFFIDNIPYSMEYDILIRYEPQLPDHWEKAVITVQRP
GRIPTSSRCGNTIPDDDNQVVSLSPGSRYVVLPRPVCFEKGTNYTVRLELPQYTSSDS
DVESPYTLIDSLVLMPYCKSLDIFTVGGSGDGVVTNSAWETFQRYRCLENSRSVVKT
PMTDVCRNIIFSISALLHQTGLACECDPQGSLSSVCDPNGGQCQCRPNVVGRTCNRC
APGTFGFGPSGCKPCECHLQGSVNAFCNPVTGQCHCFQGVYARQCDRCLPGHWGFP
SCQPCQCNGHADDCDPVTGECLNCQDYTMGHNCERCLAGYYGDPIIGSGDHCRPCP
CPDGPDSGRQFARSCYQDPVTLQLACVCDPGYIGSRCDDCASGYFGNPSEVGGSCQP
CQCHNNIDTTDPEACDKETGRCLKCLYHTEGEHCQFCRFGYYGDALQQDCRKCVC
NYLGTVQEHCNGSDCQCDKATGQCLCLPNVIGQNCDRCAPNTWQLASGTGCDPCN
CNAAHSFGPSCNEFTGQCQCMPGFGGRTCSECQELFWGDPDVECRACDCDPRGIETP
QCDQSTGQCVCVEGVEGPRCDKCTRGYSGVFPDCTPCHQCFALWDVIIAELTNRTH
RFLEKAKALKISGVIGPYRETVDSVERKVSEIKDILAQSPAAEPLKNIGNLFEEAEKLI
KDVTEMMAQVEVKLSDTTSQSNSTAKELDSLQTEAESLDNTVKELAEQLEFIKNSDI
RGALDSITKYFQMSLEAEERVNASTTEPNSTVEQSALMRDRVEDVMMERESQFKEK
QEEQARLLDELAGKLQSLDLSAAAEMTCGTPPGASCSETECGGPNCRTDEGERKCG
GPGCGGLVTVAHNAWQKAMDLDQDVLSALAEVEQLSKMVSEAKLRADEAKQSAE
DILLKTNATKEKMDKSNEELRNLIKQIRNFLTQDSADLDSIEAVANEVLKMEMPSTP
QQLQNLTEDIRERVESLSQVEVILQHSAADIARAEMLLEEAKRASKSATDVKVTADM
VKEALEEAEKAQVAAEKAIKQADEDIQGTQNLLTSIESETAASEETLFNASQRISELER
NVEELKRKAAQNSGEAEYIEKVVYTVKQSAEDVKKTLDGELDEKYKKVENLIAKKT
EESADARRKAEMLQNEAKTLLAQANSKLQLLKDLERKYEDNQRYLEDKAQELARL
EGEVRSLLKDISQKVAVYSTCL
SEQUENCE ID NO: 5
(SEQ ID NO: 5)
UGDH/Homo sapiens/Human
MFEIKKICCIGAGYVGGPTCSVIAHMCPEIRVTVVDVNESRINAWNSPTLPIYEPGLKE
V
VESCRGKNLFFSTNIDDAIKEADLVFISVNTPTKTYGMGKGRAADLKYIEACARRIVQ
NS
NGYKIVTEKSTVPVRAAESIRRIFDANTKPNLNLQVLSNPEFLAEGTAIKDLKNPDRV
LI
GGDETPEGQRAVQALCAVYEHWVPREKILTTNTWSSELSKLAANAFLAQRISSINSIS
AL
CEATGADVEEVATAIGMDQRIGNKFLKASVGFGGSCFQKDVLNLVYLCEALNLPEV
ARYWQQVIDMNDYQRRRFASRIIDSLFNTVTDKKIAILGFAFKKDTGDTRESSSIYISK
YLMDEGAHLHIYDPKVPREQIVVDLSHPGVSEDDQVSRLVTISKDPYEACDGAHAV
VICTEWDMFKELDYERIHKKMLKPAFIFDGRRVLDGLHNELQTIGFQIETIGKKVSSK
RIPYAPSGEIPKFSLQDPPNKKPKV
SEQUENCE ID NO: 6
(SEQ ID NO: 6)
ANXA2/Homo sapiens/Human
MSTVHEILCKLSLEGDHSTPPSAYGSVKAYTNFDAERDALNIETAIKTKGVDEVTIVN
IL
TNRSNAQRQDIAFAYQRRTKKELASALKSALSGHLETVILGLLKTPAQYDASELKAS
MKGLGTDEDSLIEIICSRTNQELQEINRVYKEMYKTDLEKDIISDTSGDFRKLMVALA
KGRRAEDGSVIDYELIDQDARDLYDAGVKRKGTDVPKWISIMTERSVPHLQKVFDR
YKSYSPYDMLESIRKEVKGDLENAFLNLVQCIQNKPLYFADRLYDSMKGKGTRDKV
LIRIMVSRSEVDMLKIRSEFKRKYGKSLYYYIQQDTKGDYQKALLYLCGGDD
SEQUENCE ID NO: 7
(SEQ ID NO: 7)
S100A11/Homo sapiens/Human
MAKISSPTETERCIESLIAVFQKYAGKDGYNYTLSKTEFLSFMNTELAAFTKNQKDPG
VL
DRMMKKLDTNSDGQLDFSEFLNLIGGLAMACHDSFLKAVPSQKRT
SEQUENCE ID NO: 8
(SEQ ID NO: 8)
CTSB/Homo sapiens/Human
MWQLWASLCCLLVLANARSRPSFHPLSDELVNYVNKRNTTWQAGHNFYNVDMSY
LKRLCGTFLGGPKPPQRVMFTEDLKLPASFDAREQWPQCPTIKEIRDQGSCGSCWAF
GAVEAISDRICIHTNAHVSVEVSAEDLLTCCGSMCGDGCNGGYPAEAWNFWTRKGL
VSGGLYESHVGCRPYSIPPCEHHVNGSRPPCTGEGDTPKCSKICEPGYSPTYKQDKHY
GYNSYSVSNSEKDIMAEIYKNGPVEGAFSVYSDFLLYKSGVYQHVTGEMMGGHAIR
ILGWGVENGTPYWLVANSWNTDWGDNGFFKILRGQDHCGIESEVVAGIPRTDQYW
EKI
SEQUENCE ID NO: 9
(SEQ ID NO: 9)
TP53/Homo sapiens/Human
MEEPQSDPSVEPPLSQETFSDLWKLLPENNVLSPLPSQAMDDLMLSPDDIEQWFTEDP
GP
DEAPRMPEAAPPVAPAPAAPTPAAPAPAPSWPLSSSVPSQKTYQGSYGFRLGFLHSG
TAKSVTCTYSPALNKMFCQLAKTCPVQLWVDSTPPPGTRVRAMAIYKQSQHMTEV
VRRCPHHERCSDSDGLAPPQHLIRVEGNLRVEYLDDRNTFRHSVVVPYEPPEVGSDC
TTIHYNYMCNSSCMGGMNRRPILTIITLEDSSGNLLGRNSFEVRVCACPGRDRRTEEE
NLRKKGEPHHELPPGSTKRALPNNTSSSPQPKKKPLDGEYFTLQIRGRERFEMFRELN
EALELKDAQAGKEPGGSRAHSSHLKSKKGQSTSRHKKLMFKTEGPDSD
SEQUENCE ID NO: 10
(SEQ ID NO: 10)
MGMT/Homo sapiens/Human
MDKDCEMKRTTLDSPLGKLELSGCEQGLHEIKLLGKGTSAADAVEVPAPAAVLGGP
EPLMQCTAWLNAYFHQPEAIEEFPVPALHHPVFQQESFTRQVLWKLLKVVKFGEVIS
YQQLAALAGNPKAARAVGGAMRGNPVPILIPCHRVVCSSGAVGNYSGGLAVKEWL
LAHEGHRLGKPGLGGSSGLAGAWLKGAGATSGSPPAGRN
```

1. A method of therapeutically treating brain cancer or CNS cancer comprising administering an effective amount of hydroxyureamethyl-acylfulvene to a subject in need thereof.

2. The method of claim 1, wherein the brain cancer is a glioblastoma multiforme.

3. The method of claim 1, wherein the additional therapeutic agent is selected from the group consisting of temozolomide, bevacizumab, everolimus, carmustine, lomustine, procarbazine, vincristine, irinotecan, cisplatin, carboplatin, methotrexate, etoposide, vinblastine, bleomycin, actinomycin, cyclophosphamide, and ifosfamide.

4. The method of claim, wherein the additional therapeutic agent is selected from the group consisting of cisplatin, paclitaxel, and other available therapies.

5. The method of claim 1, further comprising subjecting the subject to radiation therapy.

6. The method of claim 1, wherein the radiation therapy is selected from whole-brain irradiation, fractionated radiotherapy, radio surgery, and a combination thereof.

7. The method of claim 1, wherein the cell is in a subject is an animal.

8. The method of claim 1, wherein the subject or mammal is a human.

9. The method of claim 1, further comprising measuring expression of genetic information of brain cancer to determine if the expression of the genetic information is greater than or less than a reference level of the genetic information.

10. The method of claim 8, wherein the genetic information is EGFR, NF1, PDGFRA, or IDH1.

11. The method of claim 8, wherein the genetic information is LAMB1, UGDH, ANXA2, S100A11, and CTSB.

12. The method of claim 8, wherein the genetic information is high-level EGFR amplification, deletions in NF1 gen, high expression of MET gene, alternations of PDFRA gene, point mutations in IDH1, TP53 mutation, expression of neuron markers by astrocytes, mutations in oncogenes, MGMT methylation, or a combination thereof.

13. The method of claim 1, further comprising measuring the expression of MGMT+ or MGMT−.

14. The method of claim 14, wherein the characterization further comprises a molecular subtype classification as a Classical, Mesenchymal, Proneural, or Neural glioblastoma.

15. A method of therapeutically treating brain cancer or CNS cancer in a subject comprising:
obtaining a tumor sample from the subject;
measuring the expression of MGMT+ or MGMT− in the tumor sample; and
administering an effective amount of hydroxyureamethyl-acylfulvene or a pharmaceutically acceptable salt thereof to a subject in need thereof when MGMT is measured to be greater than a reference, wherein the reference is the level of MGMT in a healthy person.

16. The method of claim 15, wherein the brain cancer further comprises a molecular subtype classification as a Classical, Mesenchymal, Proneural, or Neural glioblastoma.

17. A method of treating brain cancer, comprising
a) detecting, in a human subject, the presence of a genetic information;
b) administering hydroxyureamethyl-acylfulvene or a pharmaceutically acceptable salt thereof, to the subject;
c) obtaining a tumor sample from the subject after administering the hydroxyureamethyl-acylfulvene or a pharmaceutically acceptable salt thereof;
d) measuring the level of a marker in the tumor sample compared to a reference level, wherein the reference is the level of MGMT in a healthy person; and
e) continuing to administer the hydroxyureamethyl-acylfulvene or a pharmaceutically acceptable salt thereof to the subject when the mutation is detected.

19. The method of claim 18, wherein the marker is substantially similar to EGFR (SEQ ID NO: 1), NF1 (SEQ ID NO: 2), IDH1 (SEQ ID NO: 3), LAMB1 (SEQ ID NO: 4), UGDH (SEQ ID NO: 5), ANXA2 (SEQ ID NO: 6), S100A11 (SEQ ID NO: 7), CTSB (SEQ ID NO: 8), TP53 (SEQ ID NO: 9), and MGMT (SEQ ID NO: 10).
